(19) United States
(12) Patent Application Publication
(10) Pub. No.: US 2026/0315078 A1
(43) Pub. Date: Oct. 15, 2026

(54) WEAPON SLING WITH INTEGRATED TOURNIQUET FUNCTIONALITY

(71) Applicant: Nicholas Andrew Gardner, La Crosse, WI (US)

(72) Inventor: Nicholas Andrew Gardner, La Crosse, WI (US)

(21) Appl. No.: 19/645,408

(22) Filed: Apr. 13, 2026

Related U.S. Application Data
(60) Provisional application No. 63/787,943, filed on Apr. 13, 2025.

(51) Int. Cl. A61B 17/132; F41C 33/00

(52) U.S. Cl. CPC A61B 17/1327; F41C 33/002

(57) ABSTRACT

A multifunctional sling for a firearm configured to function as an emergency tourniquet is disclosed. The system includes a primary sling body, a narrowing webbing section configured to interface with a windlass, and a securing section configured to retain the windlass in a stowed or tensioned position. A one-directional tensioning device enables initial tightening of a loop around a limb, and a windlass provides additional constriction through rotation. Retention features secure the windlass after tightening. The sling maintains standard firearm carrying functionality while enabling rapid conversion into a tourniquet without requiring removal from the user's equipment and is configured for one-handed deployment.

### FIELD OF THE INVENTION

The present invention relates generally to firearm slings and, more particularly, to a sling incorporating integrated hemorrhage control functionality.

### BACKGROUND OF THE INVENTION

Severe extremity hemorrhage remains a leading cause of preventable death in both civilian and tactical environments. Tourniquets are effective but are often not immediately accessible when needed.

Firearm slings are consistently carried and attached to the user's weapon system. However, conventional slings provide no medical capability.

Accordingly, there exists a need for a system that integrates hemorrhage control functionality into equipment that is always present on the user.

### SUMMARY OF THE INVENTION

The present invention provides a sling system that functions both as a firearm support device and as a rapidly deployable tourniquet.

The system includes a primary sling body, a reduced-width webbing section configured to interact with a windlass, a one-directional tensioning device for initial tightening, and a securing section configured to retain the windlass. The device is configurable between a firearm support configuration and a tourniquet configuration and is operable with one hand.

### BRIEF DESCRIPTION OF THE DRAWINGS

FIG. 1 is a close-up view of a tourniquet mechanism of the sling.

FIG. 2 is a plan view of the sling assembly illustrating component relationships.

FIG. 3 is a side view illustrating loop formation and sling geometry.

### DETAILED DESCRIPTION OF THE INVENTION

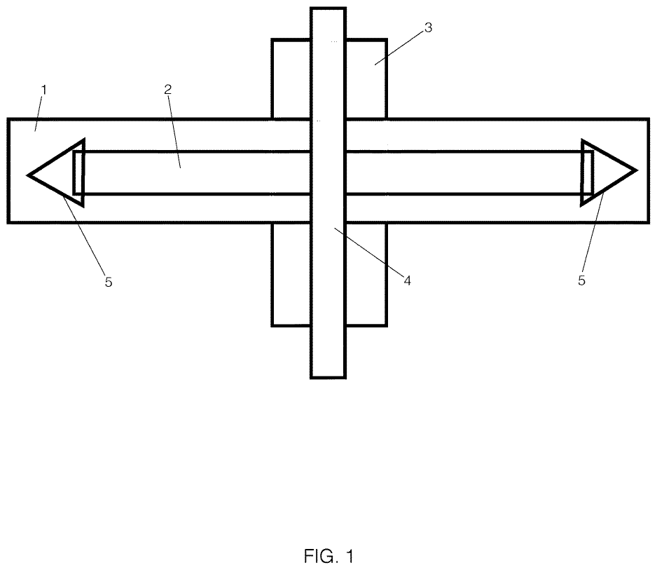

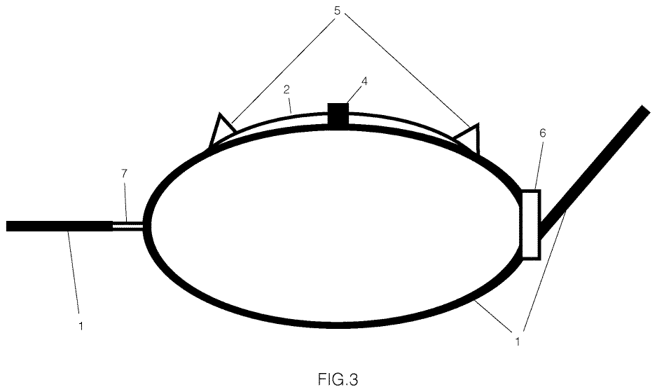

Referring to FIGS. 1-3, the invention comprises a multifunctional sling device incorporating integrated tourniquet functionality.

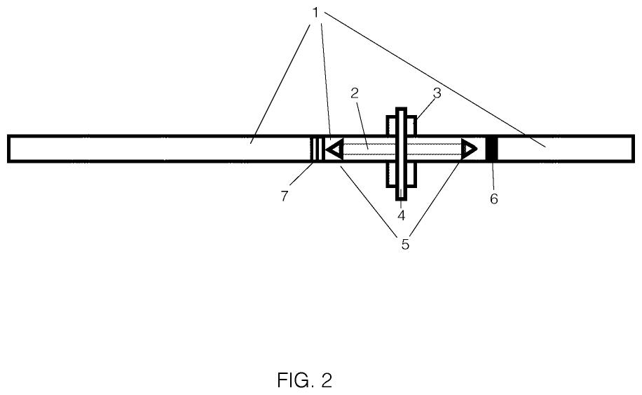

As shown in FIG. 2, the device includes a main sling body (1), which is formed from a flexible, high-strength material and configured to support a firearm during normal use.

A narrower webbing section (2) extends from or is integrated with the main sling body (1). This narrower section (2) is configured to pass through and interact with a windlass (4), allowing rotational tightening of the system. The reduced width facilitates efficient winding and increased mechanical advantage during windlass operation.

A securing webbing section (3) is positioned adjacent to the windlass (4) and is configured to retain the windlass in a stowed configuration during normal sling use and in a secured configuration after tourniquet application.

The windlass (4) is an elongated rigid member configured to be rotated to apply circumferential constriction force to the loop formed by the sling. The windlass may include a slot or aperture for receiving the narrower webbing section (2).

A pair of retention structures, illustrated as triangle cleats (5), are positioned to receive and hold the windlass (4) after rotation, thereby preventing unwinding and maintaining applied pressure.

A one-directional tensioning device, illustrated as a cam buckle (6), is configured to allow tightening of the sling in one direction while resisting loosening in the opposite direction. This enables rapid initial constriction of the loop prior to windlass use.

A tri-glide (7) or similar adjustment component is included to manage webbing routing and provide additional adjustment functionality.

In operation, the device transitions from a firearm support configuration to a tourniquet configuration by forming a loop around a limb using the sling body (1), tightening the loop using the cam buckle (6), and further constricting the loop by rotating the windlass (4). The windlass (4) is then secured within the triangle cleats (5).

The device is configured to allow one-handed deployment, enabling self-application in emergency situations.

The system may be used with the sling fully attached to a firearm or may involve partial detachment depending on user preference and configuration.

Alternative embodiments may include variations in windlass shape, retention mechanisms, tensioning devices, and attachment interfaces without departing from the scope of the invention.

1. A multifunctional sling device comprising:
a main sling body (1) formed from flexible material and configured to support a firearm;
a narrower webbing section (2) configured to pass through a windlass;
a loop portion formed by the sling and configured to encircle a limb;
a one-directional tensioning device (6) configured to tighten the loop;
a windlass (4) configured to apply additional constriction force through rotation; and
a securing structure (3, 5) configured to retain the windlass in a tightened position;
wherein the sling is configurable between a firearm support configuration and a tourniquet configuration.

2. The device of claim 1, wherein the narrower webbing section (2) is reduced in width relative to the main sling body (1).

3. The device of claim 1, wherein the windlass (4) includes a slot configured to receive the narrower webbing section (2).

4. The device of claim 1, wherein the securing structure includes at least one cleat (5).

5. The device of claim 1, wherein the securing structure further includes a webbing section (3) configured to retain the windlass.

6. The device of claim 1, wherein the one-directional tensioning device (6) comprises a cam buckle.

7. The device of claim 1, further comprising a tri-glide (7) configured to guide webbing.

8. The device of claim 1, wherein the windlass (4) has a non-circular cross-section.

9. The device of claim 1, wherein the sling is configured for one-handed tourniquet application.

10. The device of claim 1, wherein the loop portion is dynamically formed during deployment.

11. A method of controlling hemorrhage using a sling device comprising:
forming a loop around a limb using a sling body; tightening the loop using a one-directional tensioning device; rotating a windlass coupled to a narrower webbing section; and securing the windlass using a retention structure.

12. The method of claim 11, wherein the method is performed using one hand.

13. The method of claim 11, further comprising partially detaching the sling from a firearm prior to tightening.

14. A system comprising:
a firearm; and
a sling coupled to the firearm, the sling including:
a main sling body (1);
a narrower webbing section (2);
a windlass (4);
a one-directional tensioning device (6); and
a securing structure (3, 5);
wherein the sling is configurable for use as a tourniquet.

15. The system of claim 14, wherein the sling remains attached to the firearm during tourniquet application.

16. The system of claim 14, wherein the sling includes quick-detach attachment interfaces.

17. The system of claim 14, wherein the sling is configured for one-handed deployment.

18. The device of claim 1, wherein the securing structure inhibits rotational unwinding of the windlass.

19. The device of claim 1, wherein the narrower webbing section increases tightening efficiency of the windlass.

20. The device of claim 1, wherein the sling is configured to transition between configurations without full removal from a user.
